(19) United States
(12) Patent Application Publication
(10) Pub. No.: US 2006/0141017 A1
(43) Pub. Date: Jun. 29, 2006

(54) Zinc-based compositions and methods of use

(76) Inventors: William O. Kling, Dallas, TX (US); Laura K. S. Parnell, Missouri City, TX (US)

Correspondence Address: MORGAN LEWIS & BOCKIUS LLP, 1111 PENNSYLVANIA AVENUE NW, WASHINGTON, DC, 20004, US

(21) Appl. No.: 11/317,923

(22) Filed: Dec. 23, 2005

Related U.S. Application Data
(60) Provisional application No. 60/639,040, filed on Dec. 23, 2004.

(51) Int. Cl. A61K 33/32; A61L 15/00

(52) U.S. Cl. 424/445; 424/641

(57) ABSTRACT

The invention relates generally to zinc-based compositions for topical use in humans and animals, and methods therefor. The invention further relates to the use of zinc-based compositions in wound treatment, cleaning and other therapeutic applications.

### CROSS REFERENCES TO RELATED APPLICATIONS

This application claims the benefit of U.S. Provisional Application No. 60/639,040, filed Dec. 23, 2004.

### BACKGROUND OF THE INVENTION

1. Technical Field of the Invention

The invention relates generally to zinc-based compositions for use in antimicrobial treatments and methods for using these compositions. The invention relates more specifically to the use of zinc-based compositions suitable for topical application in humans and animals.

2. Description of the Prior Art

In a time when microbial resistance is a constant threat, the need for new antimicrobials is greater than ever. Topical antimicrobials are used on wounded skin to prevent microbes from invading the wound. Once microbes invade a wound, the detrimental effects can range from a delay in healing, to death by sepsis. In addition, topical antimicrobial hand-washes are used on intact skin by consumers for self-protection and by medical staff to protect themselves and patients from transferred microbes.

Some heavy metals are known to exert antimicrobial effects. Silver-based dressings are currently used as wound dressings to reduce the microbial burden of the wound. Zinc is a metal that has been purported to have antimicrobial properties. Upon close review of the literature however, not all products containing zinc have been consistent at reducing the microbial burden or aiding in the healing of wounds. There are several articles in which safety, absorption, and activity have been called into question. Review of the published literature and prior art indicates that products capable of consistently producing an antimicrobial affect are either drugs such as sulfadiazine or pyrithione that are combined with zinc, or other antimicrobial compounds in combination with zinc. Use of these compositions can be problematic for two reasons. First, when drugs are used topically, the majority of them will cause an increase in bacterial resistance to the drug component. Secondly, many antimicrobial compounds can act as an irritant and can sensitize people. Upon application of the compound following sensitization, the reaction can range from raised whelps to anaphylactic shock, which in turn can lead to death.

Metals such as aluminum, barium, beryllium, bismuth, cadmium, calcium, chromium, cobalt, copper, gallium, germanium, gold, indium, iron, lead, magnesium, manganese, molybdenum, nickel, palladium, platinum, scandium, silver, strontium, tin, titanium, vanadium, and zinc have varying antimicrobial affects. Each element has advantages and disadvantages. Some are antimicrobial, but extremely toxic. Others have a good antimicrobial spectrum, but are expensive and/or rare. Calcium, chromium, copper, iron, magnesium, manganese, and zinc are all known to be used within the body and would be logical choices to include in testing.

There is therefore a need for antimicrobial and cleaning agents that can be used topically without resulting in adverse effects to user. Zinc and zinc salts have an excellent safety profile, do not cross the blood brain barrier, can be eliminated by the body, and pose little risk, thereby making zinc an agent of choice for reducing microbes in humans and animals. In addition, zinc is an essential nutrient that has been implicated in promoting healing in elderly and zinc-deficient persons, and thus its use in such persons would serve more than one benefit.

### SUMMARY OF THE INVENTION

The invention relates generally to zinc-based compositions for use in antimicrobial and cleaning treatments and methods for using these compositions.

The invention further relates to zinc-based compositions that can be topically applied to a surface on humans or animals, for the purpose of killing existing microbes, preventing their spread to other surfaces and inhibiting future growth on the surface following application of the zinc-based compositions.

An aspect of the invention further provides a method for treating wounds by applying zinc-based compositions to the wound, which in turn promote healing of the wound.

### DESCRIPTION OF ILLUSTRATIVE EMBODIMENTS

The inventive compositions described herein have consistently exhibited antimicrobial activity, which does not appear to be related to the coupling compounds that may be additionally present in the compositions. Because of these results, the antimicrobial activity can be attributed to the zinc itself. In an embodiment of the invention, a blend of zinc salts, colloidal zinc, zinc ligands, zinc alloys, or other zinc compounds with various dissociation rates are used to make solutions, gels or wound dressings of various design. The varying dissociation rates are important for ensuring constant antimicrobial activity during the period of application of zinc-based composition.

In vitro studies have been performed to optimize and ascertain the antimicrobial activity of the invention. Contrary to anecdotal data found in the literature, formulations of the invention have been shown to exhibit antimicrobial activity against a variety of microbes including, but not limited to, bacteria, fungi, molds, yeast and viruses. In addition, the compositions of the invention are efficacious against other types of microbes including, but not limited to, algae and protozoa.

Tests have been performed in vivo and in vitro to determine the cytotoxic effects of the compositions of the invention. The tests indicate that the compositions of the claimed invention exhibit minimal levels of cytotoxicity and thus are suitable and safe for use in humans and animals.

Embodiments of the invention are useful as a wound dressing. Specifically, compositions of the invention can be immobilized on gauze, bandages, cloth, composites or other applicators, or within viscous vehicles or as a solution, and placed either temporarily or for an extended length of time on the surface of a subject having at least one wound. As defined herein, a wound is an injury, in which the skin or another external surface is torn, pierced, abraded, cut, or otherwise broken or injured. As referred to herein, the term wound is also intended to cover openings and cuts that are created on the surface, including oral and ocular surfaces, of a human or animal during surgical procedures. Compositions of the invention are suitable for use on a wound that is acute (quick healing) as well as a wound that is chronic (delayed healing).

An embodiment of the invention is directed to a method for treating ailments of intact or injured external surfaces (such as skin) or intact or injured mucous membranes of a subject comprising applying a therapeutically effective amount of an anti-microbial, zinc-based composition to at least a portion of the intact or injured surface or intact or injured mucous membranes of the subject.

Certain embodiments of the zinc-based compositions of the invention are useful for topical application on a surface of a human or animal. Compositions of the invention can be safely applied on the skin, hair, nails of animals and humans. In addition, the zinc-based compositions can be used on the surface of the eye and in the ocular cavity, for example in the form of ointment, drops or dressing. The zinc-based compositions can also be used on the surface of teeth and gum, for example during dental procedures, as well as in other areas of the oral cavity, such as the inner recesses of the mouth. Specific applications include the use of zinc-based compositions for irrigation of the mouth during dental surgery and other procedures where microbial contamination is a concern.

In an embodiment of the invention, a zinc-based agent is used to ameliorate an ailment on the surface of a human or animal subject by topical or surface application of the agent to an area of occurrence of the ailment. As used herein, the term surface when used in the context of a human or animal subject is intended to cover the external surface of the subject, such as skin, hair and nails, and further includes internal surfaces of the subject's body that are accessible for application of the zinc-based compositions including, but not limited to, mucous membranes, gum, teeth, tongue, tongue, oral cavity, ear canal, ocular cavity, urinary tract, vaginal canal and anal canal.

The term ailment encompasses all conditions that exhibit surface symptoms including, but not limited to, (i) ailments caused by external agents such as sun burn (exposure to sun) and frost bite (exposure to cold), (ii) ailments such as ringworm and eczema that are caused by microbes but manifest symptoms on the external surface of the human or animal subject, (iii) ailments that arise as a result of autoimmune diseases such as lupus that also exhibit surface symptoms, and (iv) ailments that result from surgical intervention.

Compositions of the invention can be used as a lavage or irrigant for procedures such as preoperative and postoperative reduction of bacterial load. Current preoperative technique consists of scrubbing, washing, or “painting” tissue with betadine iodine (cytotoxic antimicrobial) or alcohol (irritating and drying) or chlorhexidine gluconate (irritating, drying and requires rinsing) prior to procedure. Postoperative technique usually consists of irrigating the tissue with saline (nonantimicrobial) or betadine iodine (cytotoxic antimicrobial, skin discolorization). Specific procedures that require reduction of bacterial load to prevent contamination would be donor site harvesting, skin grafting, and meshed skin grafting. Dental procedures such as placement of crowns, root canal, or dental implants would all benefit from reduced bacterial loads. Gynecological and obstetrical procedures such as colposcopy, intrauterine device placement, childbirth, episiotomy require use of antimicrobials because of the natural environment is conducive to microbial growth. Urinary, proctology and rectal procedures such as hemorrhoidectomy, hemorrhoidopexy, prostate irradiation implantation, and catherization have a high risk of microbial contamination. All of the aforementioned situations represent potential areas of application for the zinc-based compositions of the invention.

In certain embodiments of the invention, a zinc-based composition may be topically applied to a surface of a human or animal in order to treat ailments such as itchiness, flaking, redness and other similar ailments of the skin. In certain embodiments of the invention, the topical application of the zinc-based composition occurs on the skin of a human or animal subject. Certain embodiments of the invention can be used to alleviate the effects of an itchy scalp and dandruff. Other aspects of the invention may be used to stop or prevent the growth of nail fungus.

An embodiment of the invention includes the use of a zinc-based composition in a hand cleanser or hand sanitizer. Currently available hand sanitizers in the market place have high alcohol content and are thus flammable and are toxic and thus must be kept out of the reach of children. The high levels of alcohol remove the microbes, but also the natural antimicrobial oils and compounds produced by the skin thus drying the skin. This dryness actually leaves the skin at an increased risk of infection because the natural antimicrobial compounds are removed and the skin dryness allows cracks and fissures to occur, opening new areas for point of entry for micro-organisms.

The antimicrobial properties of zinc compounds make them useful for inclusion in hand cleansers and hand sanitizers that can be used without needing to be washed off. Additionally, zinc compounds may be used in cleaning applications without the fear of any accompanying cytotoxic effects. As used herein, the term “cleaning” is intended to refer to the removal of dirt and microbes from a surface, for example, a hand, such that the surface is cleaned and sanitized following application of a cleaning agent. Cleaning applications of the compositions of the invention can be directed to intact or injured external surfaces (such as skin) or intact or injured mucous membranes.

A further embodiment of the invention is directed to a method for treating a wound, comprising, a) providing: (i) a treatment-inducing agent comprising at least one zinc compound, and (ii) a subject having at least one wound; and b) administering said treatment-inducing agent to said subject under conditions such that the healing of said wound is promoted. In the promotion of wound healing, the zinc compound (i) can function as an antimicrobial agent, (ii) restore zinc levels when applied to zinc-deficient areas that retard healing the healing process, (iii) optimize the level of moisture at the location of application thereby promoting wound healing, or (iv) effectuate various cellular and enzymatic pathways in the cells and tissue surrounding the area of application to promote the healing process. In certain embodiments of the invention, the zinc compound participates in one or more of the above functions in promoting wound healing.

The methods of the invention are applicable to patients suffering from pressure or decubitus ulcers, venous stasis ulcers, diabetic ulcers, arterial ulcers, chemical, thermal or electrical burns, skin grafts, donor sites, sclerosis, dermatitis, cuts, abrasions, denuded tissue, canker sores, tissue biopsy, surgical incisions, tissue debridement, dehiscent wounds, or other impairment of skin.

The methods of the claimed invention may be practiced with compositions comprising one or more zinc compounds. The zinc compounds include, but are not limited to, zinc salts such as zinc acetate, zinc butyrate, zinc chloride, zinc citrate, zinc gluconate, zinc glycerate, zinc glycolate, zinc formate, zinc lactate, zinc phthalocyanine, zinc picolinate, zinc proprionate, zinc salicylate, zinc tartrate and zinc undecylenate.

In certain embodiments of the invention, the zinc compound is a zinc alloy. In other embodiments of the invention, the zinc compound is colloidal zinc or a zinc ligand in a metalloenzyme. The metalloenzymes that are capable are being used in the embodiments of the invention include, but are not limited to, tissue inhibitor matrix metalloproteinase (TIMP) and porphyrins, as well as zinc ligands such as zinc bound to specific antibodies, cytokines, hormones and enzymes that effectuate and catalyze tissue and body reactions.

In an embodiment of the invention, the zinc-based compositions of the invention comprise between 0.8% (w/w) to 15% (w/w) of a zinc compound. In certain embodiments, the zinc-based compositions of the invention comprise between 0.4% (w/w) and 25% (w/w) of at least one zinc compound. In other embodiments of the invention, the amount of a zinc compound in the compositions is at least 0.4% (w/w).

In certain embodiments, the zinc-based composition comprises between 0.4% (w/w) to 25% (w/w) of a first zinc compound and 0.01% (w/w) to 10% (w/w) of a second zinc compound. The latter concentration range of 0.01% (w/w) to 10% (w/w) applies to a second zinc compound, when the second zinc compound is present in the zinc-based compositions of the invention. When the zinc-based composition of the invention comprises only a single species or type of zinc compound, the concentration of the zinc compound is at least 0.4% (w/w).

In certain embodiments of the invention, a first zinc compound is present at a concentration of at least 0.4% (w/w), in conjunction with a second zinc compound that is present at a concentration of less than 0.4% (w/w), for e.g., 0.4% (w/w) of a first zinc compound and 0.02% (w/w) of a second zinc compound. In other embodiments of the invention, a zinc-based composition comprises a first zinc compound at a concentration of at least 0.4% (w/w) and a second zinc compound that is present at a concentration of greater than 0.4% (w/w), for e.g., 0.4% (w/w) of a first zinc compound and 0.5% (w/w) of a second zinc compound.

In certain embodiments, the zinc-based composition further comprises a carrier vehicle. The carrier vehicle is present at a concentration of between 0.01% (w/w) to 99.9% (w/w), and include, but are not limited to, water, ethanol, dimethicones, silicones, carbomer, acrylamide, polyacrylamide, or petrolatum. Certain embodiments contain between 1% (w/w) to 99% (w/w) of carrier vehicle. Other embodiments contain between 5% (w/w) and 95% (w/w) of carrier vehicle.

In certain embodiments of the invention, the zinc-based composition is a solution. The treatment-inducing agent can also take the form of a gel, aerosol, powder, emulsion, slurry, cream, lotion, bandage or dressing.

In certain embodiments, the zinc-based composition further comprises an emollient. The emollient is present at a concentration between 0.3% (w/w) and 90.0% (w/w). However, in certain embodiments of the invention, the emollient is present at a concentration of up to 98% (w/w). The emollient may be selected from the group consisting of glycerin, propylene glycol, butylene glycol, petrolatum, mineral oil, lanolin, olive oil, cocoa butter, shea butter, isopropyl palmitate, octyl stearate, isopropyl myristate, dimethicone, cyclomethicone, silicone polymers, methyl gluceth-20 benzoate, C₁₂-C₁₅ alkyl benzoate, glycol distearate, paraffin, glyceryl stearate, sodium PCA, D-panthenol, cetyl octanoate, and caprylic/capric triglycerides.

In certain embodiments, the zinc-based composition further comprises a gelling or thickening agent. The gelling or thickening agent is typically present at a concentration between 0.05% (w/w) and 10.0% (w/w). The gelling or thickening agent may be selected from the group consisting of xanthan gum, hydroxyethylcellulose, carbomer, polyether-1, starch and pectin.

In certain embodiments, the zinc-based composition further comprises a silicone polymer. The silicone polymer is present at a concentration of between 0.1% (w/w) and 10% (w/w). The silicone polymer may be selected from a group consisting of polydimethylsiloxane polymer, dimethiconol fluid in dimethicone, cyclomethicone, dimethicone copolyl, and silicone glycol.

In certain embodiments, the zinc-based composition further comprises a secondary anti-microbial agent. The secondary anti-microbial agent is typically present at a concentration of between 0.05% (w/w) and 10% (w/w). The secondary anti-microbial agent may be selected from the group consisting of one or more of chlorhexidine gluconate, benzalkonium chloride, iodopropynylbutyl carbamate, phenoxyethanol, polymyxin B, neomycin, triclosan, parachlorometaxylene, incroquat and octoxyglycerin.

Certain embodiments of the zinc-based composition further comprise a stabilizing agent. The stabilizing agent is typically present at a concentration of between 0.1% (w/w) and 1.0% (w/w), and may be either an antioxidant or a surfactant. The antioxidant may be selected from the group consisting of Vitamin C and Vitamin E. The surfactant may be selected from the group consisting of incromide and silicone-based surfactant.

In certain embodiments, the zinc-based composition further comprises one or more natural or synthetic chemicals selected from the group consisting of monoterpene hydrocarbon, sesquiterpene hydrocarbon, monoterpene alcohol, sesquiterpene alcohol, monoterpene ester, sesquiterpene ester, monoterpene ether, sesquiterpene ether, monoterpene aldehyde, sesquiterpene aldehyde monoterpene ketone, sesquiterpene ketone, monoterpene oxide, sesquiterpene oxide, almond oil, ylang-ylang oil, neroli oil, sandalwood oil, frankincense oil, peppermint oil, lavender oil, jasmine absolute, geranium oil bourbon, spearmint oil, clove oil, lemongrass oil, cedarwood oil, balsam oils, tangerine oil, 1-citronellol, a-amylcinnamaldehyde, lyral, geraniol, famesol, hydroxycitronellal, isoeugenol, eugenol, eucalyptus oil, eucalyptol, lemon oil, linoleic acid, linalool and citral.

In other embodiments, the zinc-based composition further comprises an effective amount of chlorhexidine gluconate, benzalkonium chloride and incroquat.

### EXAMPLE 1

Using an agar overlay technique, dilutions of Gram positive and Gram negative bacterial suspensions were exposed to zinc-based formulations of the invention. The zinc-based formulations of the invention comprise between 0.4% (w/w) to 15% (w/w) of at least one zinc compound. The testing procedures were carried out using different carrier techniques in order to determine the relationship between the carrier technique used. As seen below, the differences in the carrier techniques resulted in a difference in the results obtained.

A typical experimental protocol for testing the efficacy of the compositions of the invention is set forth below.

The objective of the experiment was to demonstrate that the test product shows antimicrobial properties.

Cultures of the following microorganisms are maintained as stock cultures from which working inoculum are prepared. The viable microorganisms used in this test must not be more than five passages removed from the original stock culture. For purposes of the test, one passage is defined as the transfer of organisms from an established culture to fresh medium. The organisms tested are Staphylococcus aureus and Pseudomonas aeruginosa.

The materials used are test tubes with closures, pipettes (10.0 ml and 1.0 ml), serological pipettes, 100 μl and 1000 μl eppendorf tubes, 0.85% Phosphate buffered saline or peptone water, pH 7.0-7.2, petri dishes, culture loops, and other microbiological apparatuses. The media used in these experiments is tryptic soy agar.

Accurately weigh the amount of test product used for each concentration. For each concentration, add the test product to the appropriately labeled flasks containing tryptic soy agar. Mix thoroughly.

Prepare inoculum by inoculating the surface of a suitable volume of solid agar medium from a recently grown stock culture of each of the specified microorganisms. Incubate the bacterial cultures at 35° C. +/−2° C. for approximately 48 hours under aerobic conditions. To harvest the bacterial culture, place a loop full of the test microorganisms from the plate into tube containing sterile phosphate buffered saline and vortex. Adjust the count with sterile saline or additional microorganisms so that the concentration of the inoculum level is between 10⁻⁶ and 10⁻⁷ microorganisms per milliliter of product. Determine the number of viable microorganisms in each milliliter of the inoculum suspensions by serial dilution in sterile phosphate buffered saline. Plate dilutions of 10⁻⁶ and 10⁻⁷ for the test organism. Overlay with approximately 20 ml of 45° C. Tryptic Soy Agar. Incubate for 48 hours at 35° C. +/−2 C. for the test organism. Count test organisms. Calculate the number of organisms as colony forming units per ml (cfu/ml) of inoculum.

For inoculation and plating of samples, aseptically transfer the appropriate amount for the proper dilutions of the bacterial suspension into appropriately labeled 100×15 mm petri plates in duplicate. Overlay with approximately 20 ml of 45° C. tryptic soy agar with the appropriate concentration of the test product. Gently swirl plates and allow to solidify. Incubate plates for 96 hours at 35° C.

Following incubation, the plates were read and results were recorded. Using the calculated inoculum concentration of each test microorganism, the percent kill of each microorganism for each of four concentrations of test product, i.e., 0.25% w/v, 0.5% w/v, 1% w/v and 2% w/v, was calculated. Table 1 shows the data obtained in one of several experiments performed as detailed above.

TABLE 1
S. aureus

| | | | | | Control |
|---|---|---|---|---|---|
| Dilutions | 0.25% | 0.50% | 1.00% | 2.00% | (no zinc) |
| 1.0 ml (10⁻⁶) | 0 | 0 | 0 | 0 | 227 |
| 1.0 ml (10⁻⁶) | 0 | 0 | 0 | 0 | 198 |
| 0.1 ml (10⁻⁶) | 0 | 0 | 0 | 0 | 18 |
| 0.1 ml (10⁻⁶) | 0 | 0 | 0 | 0 | 19 |
| 0.1 ml (10⁵) | 0 | 0 | 0 | 0 | 198,000 |
| 0.1 ml (10⁵) | 0 | 0 | 0 | 0 | 198,000 |
| Inhibition | 100% | 100% | 100% | 100% | 0% |

P. aeruginosa

| | | | | | Control |
|---|---|---|---|---|---|
| Dilutions | 0.25% | 0.50% | 1.00% | 2.00% | (no zinc) |
| 1.0 ml (10⁻⁶) | 0 | 0 | 0 | 0 | 102 |
| 1.0 ml (10⁻⁶) | 0 | 0 | 0 | 0 | 88 |
| 0.1 ml (10⁻⁶) | 0 | 0 | 0 | 0 | 11 |
| 0.1 ml (10⁻⁶) | 0 | 0 | 0 | 0 | 9 |
| 0.1 ml (10⁵) | 0 | 0 | 0 | 0 | 95,000 |
| 0.1 ml (10⁵) | 0 | 0 | 0 | 0 | 95,000 |
| Inhibition | 100% | 100% | 100% | 100% | 0% |

### EXAMPLE 2

Using a kill rate test, dilutions of Gram positive and Gram negative bacterial suspensions were exposed to formulations of the invention. The testing procedures were carried out using different carrier techniques in order to determine the relationship between the carrier technique used. As seen below, the differences in the carrier techniques resulted in a difference in the results obtained.

A typical experimental protocol for testing the efficacy of the compositions of the invention is set forth below.

The objective of the experiment was to demonstrate that the test product shows either bactericidal or bacteristatic properties.

Cultures of the following microorganisms are maintained as stock cultures from which working inoculum are prepared. The viable microorganisms used in this test must not be more than four passages removed from the original stock culture. For purposes of the test, one passage is defined as the transfer of organisms from an established culture to fresh medium. All transfers are counted. The organisms tested are Staphylococcus aureus, Pseudomonas aeruginosa, Esherichia coli, and Candida albicans.

The materials used are test tubes with closures, pipettes (10.0 ml and 1.0 ml), serological pipettes, 100 μl and 1000 μl eppendorf tubes, 0.85% Phosphate buffered saline or peptone water, pH 4.5-5.5, sterile 1% sodium thiosulfate, petri dishes, culture loops, and other microbiological apparatuses. The media used in these experiments is tryptic soy agar with lecithin and tween 80.

Accurately pipette the test product into into an appropriately labeled or coded test tube. Store test samples at ambient temperature.

Prepare inoculum by inoculating the surface of a suitable volume of solid agar medium from a recently grown stock culture of each of the specified microorganisms. Incubate the bacterial cultures at 35° C. +/−2 C, for approximately 96 hours under anaerobic conditions. To harvest the bacterial culture, place a loop full of the test microorganisms from the plate into tube containing sterile phosphate buffered saline and vortex. Adjust the count with sterile saline or additional microorganisms so that the concentration of the inoculum level is between 10⁻⁷ and 1⁻⁸ microorganisms per milliliter of product. Determine the number of viable microorganisms in each milliliter of the inoculum suspensions by serial dilution in sterile phosphate buffered saline: Plate dilutions of 10⁻⁶, 10⁻⁷, and 10⁻⁸ for the test organism. Overlay with approximately 20 ml of 45° C. Tryptic Soy Agar with lecithin and tween 80. Incubate for 96 hours at 35° C. +/−2 C. for the test organism. Count test organism. Calculate the number of organisms as colony forming units per ml (cfu/ml) of inoculum as follows:

$$\frac{cfu \text{/} ml ( 0.1 ml )}{9.9 ml} = cfu \text{/} ml of product$$

To inoculate and plate samples, aseptically transfer 0.1 ml of the test suspension into the appropriately labeled 9.9 ml sample of test material. The test organism is inoculated as a pure culture into a single 9.9 ml sample of test material. Thoroughly mix or stir all samples by vortex. Let stand for fifteen seconds, forty-five seconds, and ninety seconds. Remove aliquots at indicated time and transfer to 9.0 ml sterile 1% Sodium Thiosulfate. Perform serial dilutions from 10⁻¹ to 10⁻⁵. Transfer 1.0 ml of each dilution into a 100×15 mm petri plate in duplicate. Overlay with approximately 20 ml of 45° C. Tryptic Soy Agar with lecithin and tween 80. Gently swirl plates and allow to solidify. Incubate plates for 96 hours at 35° C.

Following incubation, the plates were read and results were recorded. Using the calculated inoculum concentration of each test microorganism, calculate the log reduction of each microorganism for each kill rate. Table 2 below shows the data obtained in one of several experiments performed as detailed above.

TABLE 2
S. aureus

| | | | | | Control |
|---|---|---|---|---|---|
| | 4 hr | 8 hr | 12 hr | 24 hr | (no zinc) |
| CFU/ml | <1 | <1 | <1 | <1 | 12700000 |
| Log | 7.103803721 | 7.103803721 | 7.103803721 | 7.103803721 | 0 |
| Reduction | | | | | |
| Inhibition | 100% | 100% | 100% | 100% | 0% |

P. aeruginosa

| | | | | | Control |
|---|---|---|---|---|---|
| | 4 Hr | 8 Hr | 12 Hr | 24 Hr | (no zinc) |
| CFU/ml | <1 | <1 | <1 | <1 | 600000 |
| Log | 5.77815125 | 5.77815125 | 5.77815125 | 5.77815125 | 0 |
| Reduction | | | | | |
| Inhibition | 100% | 100% | 100% | 100% | 0% |

E. coli

| | | | | | Control |
|---|---|---|---|---|---|
| | 4 Hr | 8 Hr | 12 Hr | 24 Hr | (no zinc) |
| CFU/ml | <1 | <1 | <1 | <1 | 700000 |
| Log | 5.84509804 | 5.84509804 | 5.84509804 | 5.84509804 | 0 |
| Reduction | | | | | |
| Inhibition | 100% | 100% | 100% | 100% | 0% |

C. albicans

| | | | | | Control |
|---|---|---|---|---|---|
| | 4 Hr | 8 Hr | 12 Hr | 24 Hr | (no zinc) |
| CFU/ml | <1 | <1 | <1 | <1 | 12800000 |
| Log | 6.10720997 | 6.10720997 | 6.10720997 | 6.10720997 | 0 |
| Reduction | | | | | |
| Inhibition | 100% | 100% | 100% | 100% | 0% |

### EXAMPLE 3

An in vitro biocompatibility test was performed based on the requirements of the International Organization for Standardization (ISO 10993-5). Two sample compositions were tested to determine cytotoxicity potential.

Filter disc with a 0.1 ml aliquot of sample and filter disc control with a 0.1 ml aliquot of 0.9% sodium chloride irrigation USP, a negative control of 1 cm×1 cm high density polyethylene, and a positive control of 1 cm×1 cm latex were placed on triplicate agarose surfaces directly overlaying confluent monolayers of mouse fibroblast cells.

The cell culture was propagated and maintained in open flasks containing single strength Minimum Essential Medium (MEM) supplemented with 5% serum and 2% antibiotics in a gaseous environment of 5% carbon dioxide (CO₂). Cell culture 10 cm² wells were seeded, labeled with passage number and date, and incubated at 37° C. in 5% CO₂ to obtain confluent monolayers of cells.

The growth medium in well was replaced with 2 ml of equal amounts of double strength MEM supplemented with 10% serum and 4% antibiotics, supplemented with neutral red, and 2% agarose, resulting in a final concentration of 1% agarose and single strength MEM. Then, 2 ml of MEM-agarose mixture was placed in cell culture wells and allowed to solidify over the cells to form the agarose overlay.

The filter discs and controls were placed on the solidified agarose surface in separate cell culture wells in triplicate. The wells were labeled with the lab number, date, and incubated at 37° C. in 5% CO₂ for 24 hours. Cell cultures were examined macroscopically for cell decolorization around the test article and controls to determine if zones of cell lysis were present. The cultures were then examined microscopically at 100× magnification to verify any decolorized zones and to determine cell morphology in proximity of each item.

The in vitro results were similar to other metallic agents when exposed in a static cell culture system.

An in vivo biocompatibility test was based on the requirements of the International Organization for Standardization (ISO 10993: Biological Evaluation of Medical Devices, Part 10: Tests for Irritation and Sensitization). Two sample compositions were tested to determine irritancy potential. The New Zealand white rabbit is an appropriate animal model for evaluating potential skin irritants by the current ANSI/AAMI/ISO testing standards. The rabbit is widely used for this purpose and relative ranking of irritancy scores can be determined.

All animals were housed in an AAALAC International accredited facility. Male New Zealand white rabbits were cared for by conditions set forth in the “Guide for the care and use of laboratory animals.” The animals were fed PROLAB® High fiber Rabbit Diet daily. Water was provided ad libitum and delivered through an automatic watering system. Animals were housed individually in stainless steel suspended cages and identified on the cage. Room temperature, humidity and lighting was monitored and controlled with appropriate timers.

One day prior to the treatment, the fur on each rabbit's back were clipped with an electric clipper. On the day of treatment, four sites, two on each side of the back and positioned cranially and caudally, were designated on each rabbit. The sites were free from blemishes that could interfere with the interpretation of results. Just prior to test article application, the sites on the right side of the back were abraded. Each rabbit received four parallel epidermal abrasions with a sterile needle. The sites on the left side remained intact.

Four-ply gauze (25 mm×25 mm) with 0.5 ml portions of the test samples moistened with 0.5 ml of saline and 0.5 ml saline control articles were topically applied to the cranial sites (two per rabbit). The patches were covered with polyethylene plastic backings and covered with nonreactive tape. The controls were applied similarly to the caudal sites. The back of the animal was wrapped with an elastic binder to maintain the patch position. Animals were returned to their cages after treatment.

After 24 hours, the binders, tape, and patches were removed. The sites were gently wiped with a gauze sponge dampened with deionized water in an attempt to remove any remaining residue. Dermal observations for erythema and edema were recorded at 1, 24, 48 and 72 hours after the removal of the single sample patch application.

The Primary Irritation Index of the test was calculated following test completion for each animal. The erythema and edema scores obtained at 24, 48, and 72 hour intervals were added together and divided by the total number of observations. This calculation was conducted separately for the test and control articles for each animal. The score for the control was subtracted from the score for the test article to obtain the Primary Irritation score/The Primary Irritation score for each rabbit was added together and divided by the number of rabbits to obtain the Primary Irritation Index. The Primary Irritation Index of the test sample was within the allowable range for compositions having applicability for human use.

What is claimed is:

1. A method for treating a wound, comprising:
a) providing: (i) a treatment-inducing agent comprising at least one zinc compound, and (ii) a subject having at least one wound; and
b) administering said treatment-inducing agent to said subject by application of the agent to the wound.

2. The method of claim 1, wherein said subject is a burn patient.

3. The method of claim 1, wherein said wound is a chronic wound.

4. The method of claim 1, wherein the wound is a burn wound.

5. The method of claim 1, wherein the wound is an acute wound.

6. The method of claim 1, wherein said zinc compound is a zinc salt.

7. The method of claim 1, wherein said zinc compound is a zinc alloy.

8. The method of claim 1, wherein said zinc compound is colloidal zinc.

9. The method of claim 1, wherein said zinc compound is a zinc ligand.

10. The method of claim 1, wherein said zinc compound is selected from the group consisting of zinc acetate, zinc butyrate, zinc chloride, zinc citrate, zinc gluconate, zinc glycerate, zinc glycolate, zinc formate, zinc lactate, zinc phthalocyanine, zinc picolinate, zinc proprionate, zinc salicylate, zinc tartrate and zinc undecylenate.

11. A composition for use in treating a wound, comprising a first zinc compound, wherein said zinc compound promotes healing.

12. The composition of claim 11, further comprising a carrier vehicle.

13. The composition of claim 11, further comprising an emollient.

14. The composition of claim 11, wherein said zinc compound is a zinc salt.

15. The composition of claim 11, wherein said zinc compound is a zinc alloy.

16. The composition of claim 11, wherein said zinc compound is colloidal zinc.

17. The composition of claim 11, wherein said zinc compound is a zinc ligand in a metalloenzyme.

18. The composition of claim 11, wherein said zinc compound is selected from the group consisting of zinc acetate, zinc butyrate, zinc chloride, zinc citrate, zinc gluconate, zinc glycerate, zinc glycolate, zinc formate, zinc lactate, zinc phthalocyanine, zinc picolinate, zinc proprionate, zinc salicylate, zinc tartrate and zinc undecylenate.

19. The composition of claim 11, wherein said zinc compound is present at a concentration of at least 0.4% (w/w).

20. The composition of claim 11, wherein said zinc compound is present at a concentration ranging from 0.4% (w/w) to 25% (w/w).

21. The composition of claim 11, further comprising a second zinc compound.

22. The composition of claim 21, wherein said second zinc compound is present at a concentration ranging from 0.01% (w/w) to 10% (w/w).

23. The composition of claim 12, wherein said carrier vehicle is present at a concentration of between 0.01% (w/w) to 99.9% (w/w).

24. The composition of claim 12, wherein said carrier vehicle is water.

25. The composition of claim 12, wherein said carrier vehicle is ethanol.

26. The composition of claim 11, further comprising a gelling or thickening agent.

27. The composition of claim 11, wherein said composition is a solution.

28. The composition of claim 11, wherein said composition is selected from the group consisting of a gel, aerosol, powder, emulsion, slurry, cream, lotion, bandage or dressing.

29. The composition of claim 13, wherein the emollient is present at a concentration between 0.3% (w/w) and 90.0% (w/w).

30. The composition of claim 13, wherein the emollient is present at a concentration of at least 98% (w/w).

31. The composition of claim 13, wherein said emollient is selected from the group consisting of glycerin, propylene glycol, butylene glycol, petrolatum, mineral oil, lanolin, olive oil, cocoa butter, shea butter, isopropyl palmitate, octyl stearate, isopropyl myristate, dimethicone, cyclomethicone, silicone polymers, methyl gluceth-20 benzoate, C₁₂-C₁₅ alkyl benzoate, glycol distearate, paraffin, glyceryl stearate, sodium PCA, D-panthenol, cetyl octanoate, and caprylic/capric triglycerides.

32. The composition of claim 26, wherein the gelling or thickening agent is present at a concentration between 0.05% (w/w) and 10.0% (w/w).

33. The composition of claim 26, wherein the gelling or thickening agent is selected from the group consisting of xanthan gum, hydroxyethylcellulose, carbomer, polyether-1, starch and pectin.

34. The composition of claim 11, further comprising a silicone polymer.

35. The composition of claim 34, wherein the silicone polymer is present at a concentration of between 0.1% (w/w) and 10% (w/w).

36. The composition of claim 34, wherein the silicone polymer is selected from a group consisting of polydimethylsiloxane polymer, dimethiconol fluid in dimethicone, cyclomethicone, dimethicone copolyl, and silicone glycol.

37. The composition of claim 11, further comprising a secondary anti-microbial agent.

38. The composition of claim 37, wherein the secondary anti-microbial agent is present at a concentration of between 0.05% (w/w) and 10% (w/w).

39. The composition of claim 37, wherein the secondary antimicrobial agent is selected from the group consisting of one or more of chlorhexidine gluconate, benzalkonium chloride, iodopropynylbutyl carbamate, phenoxyethanol, polymyxin B, neomycin, triclosan, parachlorometaxylene, incroquat and octoxyglycerin.

40. The composition of claim 11, further comprising a stabilizing agent.

41. The composition of claim 40, wherein said stabilizing agent is present at a concentration of between 0.1% (w/w) and 1.0% (w/w).

42. The composition of claim 40, wherein the stabilizing agent is an antioxidant or a surfactant.

43. The composition of claim 42, wherein the antioxidant is selected from the group consisting of Vitamin C and Vitamin E.

44. The composition of claim 42, wherein the surfactant is selected from the group consisting of incromide and silicone-based surfactant.

45. The composition of claim 11, which further comprises one or more natural or synthetic chemicals selected from the group consisting of monoterpene hydrocarbon, sesquiterpene hydrocarbon, monoterpene alcohol, sesquiterpene alcohol, monoterpene ester, sesquiterpene ester, monoterpene ether, sesquiterpene ether, monoterpene aldehyde, sesquiterpene aldehyde monoterpene ketone, sesquiterpene ketone, monoterpene oxide, sesquiterpene oxide, almond oil, ylang-ylang oil, neroli oil, sandalwood oil, frankincense oil, peppermint oil, lavender oil, jasmine absolute, geranium oil bourbon, spearmint oil, clove oil, lemongrass oil, cedarwood oil, balsam oils, tangerine oil, 1-citronellol, a-amylcinnamaldehyde, lyral, geraniol, famesol, hydroxycitronellal, isoeugenol, eugenol, eucalyptus oil, eucalyptol, lemon oil, linoleic acid, linalool and citral.

46. The composition of claim 11, further comprising an effective amount of chlorhexidine gluconate, benzalkonium chloride and incroquat.

47. A method for cleaning a surface of a human or animal subject, comprising:
a) providing a cleaning agent comprising at least one zinc compound; and
b) contacting said cleaning agent with the surface of the subject, wherein contacting the cleaning agent with said surface promotes cleaning of the surface.

48. The method of claim 47, wherein said zinc compound is a zinc salt.

49. The method of claim 47, wherein said zinc compound is a zinc alloy.

50. The method of claim 47, wherein said zinc compound is colloidal zinc.

51. The method of claim 47, wherein said zinc compound is a zinc ligand in a metalloenzyme.

52. The method of claim 47, wherein said zinc compound is selected from the group consisting of zinc acetate, zinc butyrate, zinc chloride, zinc citrate, zinc gluconate, zinc glycerate, zinc glycolate, zinc formate, zinc lactate, zinc phthalocyanine, zinc picolinate, zinc proprionate, zinc salicylate, zinc tartrate and zinc undecylenate.

53. The method of claim 47, wherein said surface is a skin surface.

54. The method of claim 47, wherein said surface is a mucous membrane surface.

55. The method of claim 47, wherein said surface is a hand surface.

56. The method of claim 47, wherein said surface is a facial surface.

57. The method of claim 47, wherein said surface is a nail surface.

58. The method of claim 47, wherein said surface is a scalp surface.

59. A method for ameliorating an ailment in a human or animal subject comprising:
a) providing an agent comprising at least one zinc compound;
b) applying said agent to an area of occurrence of the ailment, wherein the application of the agent to the area promotes amelioration of the ailment.

60. The method of claim 59, wherein said ailment occurs on a surface of the subject.

61. The method of claim 59, wherein said ailment occurs on the subject's skin.

62. The method of claim 59, wherein said ailment occurs on the subject's mucous membrane.

63. The method of claim 59, wherein said ailment occurs on the subject's nails.

64. The method of claim 59, wherein said ailment occurs on the subject's scalp.

65. The method of claim 59, wherein said ailment occurs on the subject's mucous membrane.

66. The method of claim 59, wherein said zinc compound is a zinc salt.

67. The method of claim 59, wherein said zinc compound is a zinc alloy.

68. The method of claim 59, wherein said zinc compound is colloidal zinc.

69. The method of claim 59, wherein said zinc compound is a zinc ligand in a metalloenzyme.

70. The method of claim 59, wherein said zinc compound is selected from the group consisting of zinc acetate, zinc butyrate, zinc chloride, zinc citrate, zinc gluconate, zinc glycerate, zinc glycolate, zinc formate, zinc lactate, zinc phthalocyanine, zinc picolinate, zinc proprionate, zinc salicylate, zinc tartrate and zinc undecylenate.
